(19) United States
(12) Patent Application Publication
(10) Pub. No.: US 2020/0146706 A1
(43) Pub. Date: May 14, 2020

(54) ULTRASONIC TREATMENT TOOL

(71) Applicant: Olympus Corporation, Tokyo (JP)

(72) Inventors: Kiichiro Sawada, Tokyo (JP); Chikayoshi Meguro, Tokyo (JP)

(73) Assignee: Olympus Corporation, Tokyo (JP)

(21) Appl. No.: 16/745,133

(22) Filed: Jan. 16, 2020

Related U.S. Application Data
(63) Continuation of application No. PCT/JP2017/026493, filed on Jul. 21, 2017.

(51) Int. Cl. A61B 17/32; A61B 17/16

(52) U.S. Cl. CPC A61B 17/320068; A61B 2017/32009; A61B 2017/320073; A61B 17/16

(57) ABSTRACT

The disclosed technology is directed to a probe unit of an ultrasonic treatment tool that comprises an ultrasonic probe having respective opposed distal and proximal end sides in a longitudinal direction and configured to transmit ultrasonic vibrations. A treatment portion is attached to the distal end side of the ultrasonic probe in the longitudinal direction. The treatment portion includes a cutting surface of a shape having directionality. A handle portion having a cutting portion identifier that is engaged with an outer circumference of the proximal end side of the ultrasonic probe. The cutting portion identifier is configured to indicate a position of a reference side representing a direction in which the cutting surface is oriented and/or included in the cutting surface.

### CROSS-REFERENCE TO RELATED APPLICATION

This application is a continuation application of PCT Application No. PCT/JP2017/026493 filed on Jul. 21, 2017, which is hereby incorporated by reference in its entirety.

### TECHNICAL FIELD

The disclosed technology relates to an ultrasonic treatment tool for performing a treatment using ultrasonic vibration.

### DESCRIPTION OF THE RELATED ART

Generally, ultrasonic treatment tools include a probe unit and a grip portion. The grip portion that is gripped by the surgeon houses therein a hollow cylindrical transducer for generating ultrasonic vibrations, and hence has an outer shape complementary to the hollow cylindrical shape of the transducer. The larger the size of the transducer is to meet demands for higher output capabilities, the more hollow cylindrical the outer shape of the grip portion becomes to prevent the grip portion from increasing in diameter.

For assembling ultrasonic treatment tools, an ultrasonic probe is threaded into a transducer in a grip portion under appropriate tightening pressure using a tool such as a torque wrench. Although the tightening pressure for tightening the screws has been optimized in the assembly process, the position where the ultrasonic probe is installed with respect to the grip portion is indefinite.

Japanese Patent No. 5784863 (PTL 1) discloses a treatment portion that has a hook-shaped treatment surface. With the hook-shaped treatment portion, the hook itself has to face the treatment surface because it cuts a treatment surface by pushing and pulling while hammering the treatment target. Although the treatment portion is attached to a hollow cylindrical grip portion, its position with respect to the grip portion is indefinite. The hollow cylindrical grip portion makes it difficult for the surgeon griping the grip portion to confirm the orientation of the treatment portion with respect to the grip portion. When the surgeon grips again the grip portion or changes the way in which it grips the grip portion, the surgeon has to directly see the orientation of the treatment portion or has to see a displayed image of the treatment portion captured by an endoscope or the like before the surgeon determines the orientation of the treatment portion. During joint surgery or the like in which the ultrasonic treatment tool is used, since the surgeon is unable to directly see the treatment portion, the surgeon needs to confirm the orientation of the treatment portion by seeing a displayed image captured by an arthroscope or endoscope. Specifically, when the surgeon has gripped again the grip portion, the surgeon has to adjust the observing direction such that the treatment portion exists in the field of vision of the arthroscope and to operate the ultrasonic treatment tool. The surgeon is thus required to finish the treatment within a predetermined period of time for the surgery by performing a complicated procedure of operating the ultrasonic treatment tool and the arthroscope.

### BRIEF SUMMARY OF EMBODIMENTS

The disclosed technology has been made in view of the problem described hereinbefore.

One aspect of the disclosed technology is directed to a probe unit of an ultrasonic treatment tool that comprises an ultrasonic probe having respective opposed distal and proximal end sides in a longitudinal direction and configured to transmit ultrasonic vibrations. A treatment portion is attached to the distal end side of the ultrasonic probe in the longitudinal direction. The treatment portion includes a cutting surface of a shape having directionality. A handle portion having a cutting portion identifier that is engaged with an outer circumference of the proximal end side of the ultrasonic probe. The cutting portion identifier is configured to indicate a position of a reference side representing a direction in which the cutting surface is oriented and/or included in the cutting surface.

Another aspect of the disclosed technology is directed to an ultrasonic treatment tool comprises a probe unit configured to be used with the ultrasonic treatment tool for treating bones and soft bones using ultrasonic vibrations. A grip portion is detachably mounted on a proximal-end side of the probe unit. The grip portion contains therein an ultrasonic transducer for producing ultrasonic waves and transmitting ultrasonic vibrations to the probe unit. The probe unit includes an ultrasonic probe having respective opposed distal and proximal end sides in a longitudinal direction and configured to transmit the ultrasonic vibrations. A treatment portion is attached to the distal end side of the ultrasonic probe in longitudinal direction. The treatment portion includes a cutting surface of a shape having directionality. A handle portion having a cutting portion identifier that is engaged with an outer circumference of the proximal end side of the ultrasonic probe. The cutting portion identifier is configured to indicate a position of a reference side representing a direction in which the cutting surface is oriented and/or included in the cutting surface.

### BRIEF DESCRIPTION OF THE DRAWINGS

The technology disclosed herein, in accordance with one or more various embodiments, is described in detail with reference to the following figures. The drawings are provided for purposes of illustration only and merely depict typical or example embodiments of the disclosed technology. These drawings are provided to facilitate the reader's understanding of the disclosed technology and shall not be considered limiting of the breadth, scope, or applicability thereof. It should be noted that for clarity and ease of illustration these drawings are not necessarily made to scale.

FIG. 1 is a view illustrating by way of example the appearance of an ultrasonic treatment tool according to an embodiment of the disclosed technology.

FIG. 2A is a view of the appearance, as viewed from above, of a probe unit of the ultrasonic treatment tool.

FIG. 2B is a view of the appearance, as viewed from one side, of the probe unit of the ultrasonic treatment tool.

FIG. 2C is a view illustrating a representation of a structure in which a probe and a handle portion are attached to one another and a fixed positional relationship between a cutting surface of a treatment portion of the probe and a marker.

FIG. 2D is a view of the appearance, as viewed from one side, of a first treatment portion.

FIG. 2E is a view of the appearance, as viewed from one side, of a second treatment portion.

FIG. 2F is a diagram illustrating a reference side of a cutting surface of the second treatment portion.

FIG. 2G is a view of the appearance, as viewed from one side, of a third treatment portion.

FIG. 3A is a view illustrating a first gripped state of a grip portion.

FIG. 3B is a view illustrating a second gripped state of the grip portion.

FIG. 3C is a view illustrating a third gripped state of the grip portion.

FIG. 3D is a view illustrating a fourth gripped state of the grip portion.

FIG. 4 is a view illustrating a marker according to a first modification on the ultrasonic treatment tool.

FIG. 5 is a view illustrating a marker according to a second modification on the ultrasonic treatment tool.

FIG. 6 is a view illustrating a second protrusion on a handle portion.

FIG. 7 is a view illustrating a protrusion formed by a first constricted portion on the handle portion.

FIG. 8 is a view illustrating a protrusion formed by a second constricted portion on the handle portion.

FIG. 9 is a view illustrating a protrusion formed by a third constricted portion on the handle portion.

### DETAILED DESCRIPTION OF THE EMBODIMENTS

In the following description, various embodiments of the technology will be described. For purposes of explanation, specific configurations and details are set forth in order to provide a thorough understanding of the embodiments. However, it will also be apparent to one skilled in the art that the technology disclosed herein may be practiced without the specific details. Furthermore, well-known features may be omitted or simplified in order not to obscure the embodiment being described.

It is an object of the disclosed technology to provide an ultrasonic treatment tool having a treatment portion whose orientation can easily be confirmed by the user for a shorter period of time required for surgery by sensing a finger touch on the ultrasonic treatment tool or seeing the hand gripping the ultrasonic treatment tool to grasp the orientation of a cutting surface of the treatment portion when the user regrasps the ultrasonic treatment tool or changes the way in which it grips the ultrasonic treatment tool.

An ultrasonic treatment tool according to embodiments of the disclosed technology will be described hereinafter with reference to the drawings.

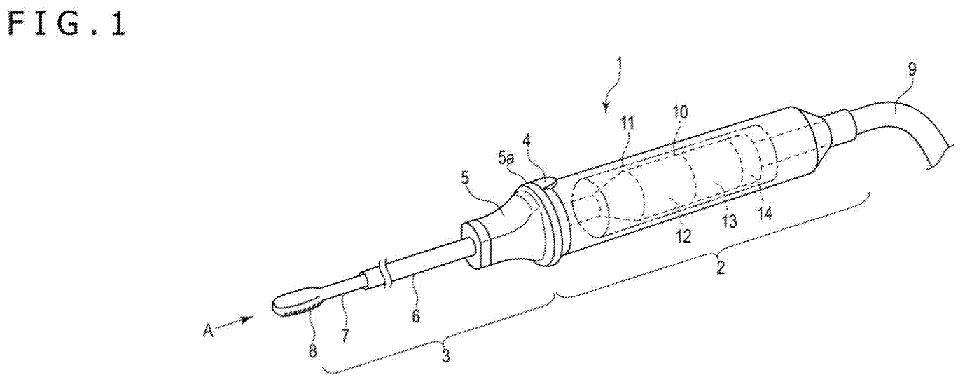

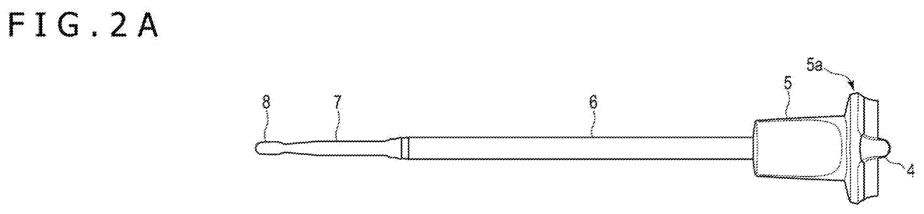

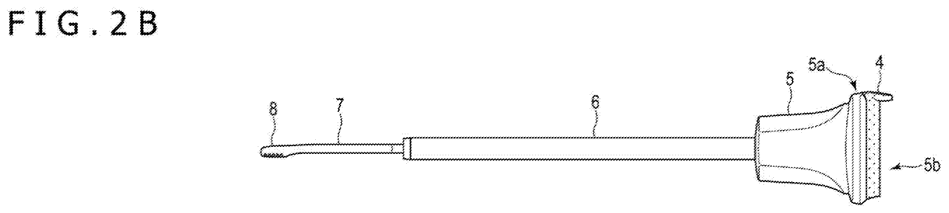

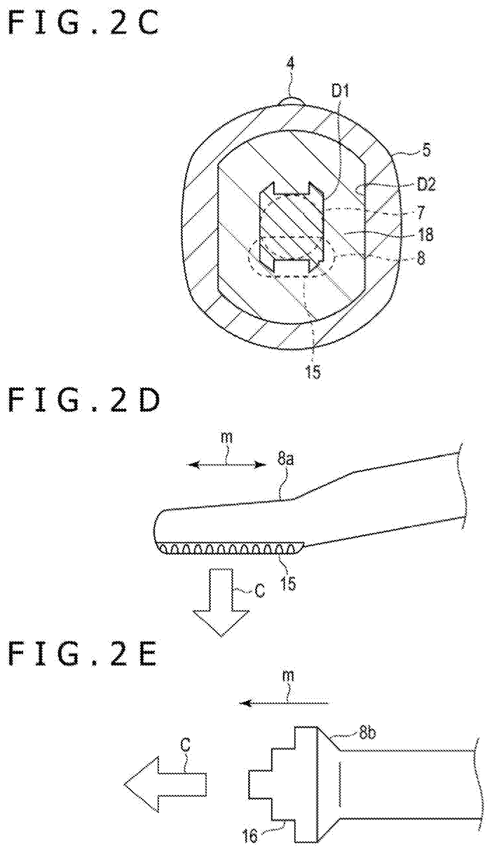

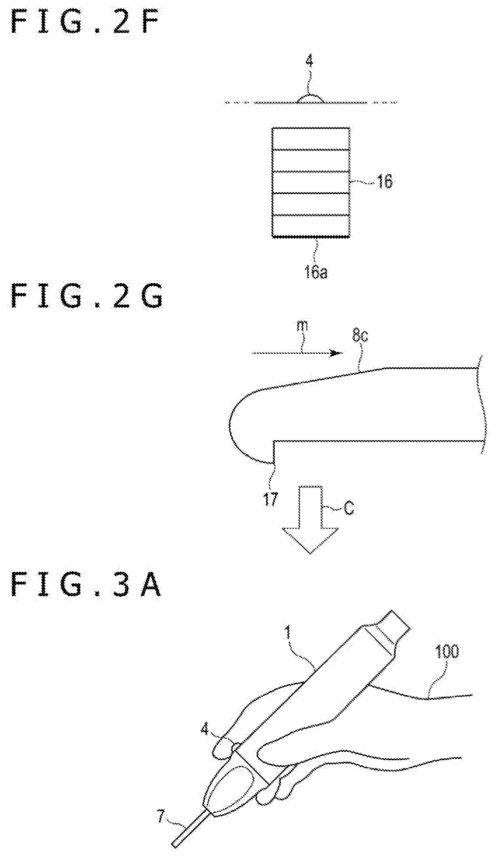

FIG. 1 is a view illustrating by way of example the appearance of an ultrasonic treatment tool, denoted by 1, according to the embodiment of the disclosed technology. FIG. 2A is a view of the appearance, as viewed from above, of a probe unit 3. FIG. 2B is a view of the appearance, as viewed from one side, of the probe unit 3. Directions and positions in the description that follows will be described hereinafter. A side of the ultrasonic treatment tool 1 where a treatment portion 8 is disposed is referred to as a distal end, i.e., distal-end side, and a side of the ultrasonic treatment tool 1 where a power supply cable 9 is disposed is referred to as a proximal end, i.e., proximal-end side. A side of a handle portion 5 and an ultrasonic probe (hereinafter referred to as “probe”) 7 where the treatment portion 8 is disposed is referred to as a distal end, i.e., distal-end side, and a side thereof that is joined to a grip portion 2 is referred to as a proximal end or a rear end. Similarly, a side of the grip portion 2 that is joined to the handle portion 5 is referred to as a distal end, i.e., distal-end side.

The ultrasonic treatment tool 1 includes the grip portion 2 that houses an ultrasonic transducer 10 therein, and the probe unit 3 that is a treatment assembly including the treatment portion 8 on its distal end. The treatment portion 8 to be described hereinafter is available in various configurations as described hereinafter. Those configurations include cutting surfaces having predetermined cutting directions and predetermined positions each serving as a reference for cutting, which may be one side of a polygonal shape if the cutting surface is polygonal in shape. The cutting surfaces with the predetermined cutting directions are said to have directionality. In order for the surgeon who uses the ultrasonic treatment tool 1 to ascertain directionality thereof, a marker, i.e., cutting portion identifier 4 to be described hereinafter suggests the direction or orientation in which the cutting surface faces.

The probe unit 3 according to the present embodiment is of the disposable type as it will not be reused. Usually, when the probe unit 3 is to be used, the probe unit 3 is taken out of a package in which it has been sterilized and sealed. A connector 12 of the grip portion 2, to be described hereinafter, and the proximal-end portion of a probe 7 of the probe unit 3 are screwed together under an appropriate tightening pressure using a torque wrench, thereby integrally combining the grip portion 2 and the probe unit 3 together.

The grip portion 2 is injection-molded into a tubular shape of a resin material, for example. The ultrasonic transducer 10 housed in the grip portion 2, which is of known nature, includes an ultrasonic vibrator 13 for producing ultrasonic vibrations and the connector 12, which is horn-shaped and connected to the probe 7, for magnifying the amplitude of the produced ultrasonic vibrations and transmitting the magnified-amplitude ultrasonic vibrations to the probe 7. The grip portion 2 also includes a power supply circuit 14 for supplying drive electric power to the ultrasonic vibrator 13. The power supply cable 9, which extends from an external power supply, not illustrated, is connected to the power supply circuit 14. The ultrasonic transducer 10 produces ultrasonic vibrations as longitudinal vibrations having an optional amplitude along its longitudinal axis.

The ultrasonic vibrator 13, the connector 12, and the power supply circuit 14 are disposed in a hollow cylindrical housing 11. The housing 11 is accommodated in the grip portion 2. In the illustrated embodiment, when an operating switch, not illustrated, on the external power supply is operated, the external power supply supplies electric power to the ultrasonic transducer 10 to enable the ultrasonic transducer 10 to produce ultrasonic vibrations. The operating switch may alternatively be mounted on the grip portion 2 or the handle portion 5.

The probe unit 3 includes the handle portion 5 fitted over the distal end of the grip portion 2, a sheath 6 having a proximal-end side attached to the handle portion 5, the probe 7 inserted in the sheath 6 and having a proximal-end side fixed to the handle portion 5, and the treatment portion 8 disposed on the distal end of the probe 7, for performing a treatment, such as cutting, on a treatment target. The handle portion 5 is of a tapered shape whose outside diameter is progressively reduced from a rear-end side thereof toward a distal-end side thereof to which the proximal-end portion of the probe 7 is fixed.

The marker, i.e., cutting portion identifier, 4 is disposed on an outer circumferential surface of the proximal-end side of the handle portion 5. In the illustrated embodiment, the marker 4 projects in a longitudinal direction from the handle portion 5 toward the proximal-end side of the grip portion 2. The marker 4 is of a color different from the handle portion 5 and the grip portion 2. The marker 4 may have surface irregularities, grooves, or the like on its surface so that the surgeon can detect the presence of the marker 4 through its tactile sense when the surgeon feels the marker 4 with a finger or a palm.

The marker 4 and the handle portion 5 are integrally formed of respective resin materials having different color by two-color injection molding, for example. Alternatively, the marker 4 and the handle portion 5 may be resin-molded separately from each other and subsequently fitted together. The marker 4 represents the orientation of a cutting surface, to be described hereinafter, of the treatment portion 8. The surgeon can recognize the orientation of the cutting surface of the treatment portion 8 with respect to the treatment target, to be described hereinafter, simply by confirming the marker 4 through a visual or tactile check. The probe 7 is attached to the handle portion 5 on its proximal-end side at a position corresponding to a node of ultrasonic vibrations, and is also supported in the sheath 6 at a position corresponding to the node of ultrasonic vibrations.

The positional relationship between a cutting surface, denoted by 15, of the treatment portion 8 of the probe 7 attached to the handle portion 5 and the marker 4 will be described hereinafter with reference to FIGS. 2C and 2D. According to the present embodiment, the known technology of D-cut using at least one flat surface, what is generally called a D-cut surface, is used as a process of fixing the probe 7 in position. As illustrated in FIG. 2C, the marker 4 and the cutting surface 15, i.e., the lower surface of an elliptical shape indicated by the dotted line, of the treatment portion 8 are disposed on respective sides that face each other across the central axis of the probe 7. In a case where the marker 4 is oriented directly upwardly, the cutting surface and a reference side of the cutting surface, to be described hereinafter, are oriented directly downwardly.

The layout in which the marker 4 and the cutting surface 15 face each other is merely an example, and they may be disposed to face in the same direction depending on the application. Specifically, in a case where the marker 4 is oriented directly upwardly, the cutting surface may be disposed to face directly upwardly. A protrusion 5a, to be described hereinafter, raises the marker 4 into a three-dimensional shape with respect to the outer circumferential surface of the grip portion 2. Accordingly, the presence of the marker 4 can be confirmed not only by a visual check, but also a finger or palm touch.

Furthermore, although the marker 4 is raised into a three-dimensional shape on the outer circumferential surface of the grip portion 2 in the present embodiment, the marker 4 may be formed as a groove cut into the apex of the protrusion 5a. At least the inside of the groove is colored differently from the handle portion 5, or a member having a color different from the handle portion 5 is fitted in the groove.

According to the present embodiment, the cutting surface 15 of the treatment portion 8a of the probe 7 and the marker 4 are disposed in facing relation to each other using two D-cut surfaces D1 on the outer circumferential surface of the proximal-end portion of the probe 7 and two D-cut surfaces D2 formed on the handle portion 5 in parallel to the D-cut surfaces D1.

First, the treatment portion 8 is disposed on the distal end of the probe 7, making the orientation of the cutting surface 15 definite. The D-cut surfaces D1 that have taken into account the orientation of the cutting surface are formed on the distal-end side of the probe 7 at a position corresponding to a node of ultrasonic vibrations. Conversely, after the D-cut surfaces D1 are formed on the probe 7 and the two D-cut surfaces D2 are formed as illustrated in FIG. 2C, the treatment portion 8 may be mounted in place at a position matching those surfaces. In the illustrated embodiment, since the D-cut surfaces are formed, at least one cutout may be formed in an end of one of the D-cut surfaces to prevent the probe 7 and the handle portion 5 from being assembled in an inversed state, i.e., a state in which they are turned 180° about the longitudinal axis.

Next, the handle portion 5 that has in its inside the D-cut surfaces D2 with the determined positional relationship to the marker 4 is injection-molded. The probe 7 is fixed to the handle portion 5 at a prescribed position using the D-cut surfaces D1 and the D-cut surfaces D2. With the arrangement illustrated in FIG. 2C, a fixing member 18 is inserted between the probe 7 and the handle portion 5 for indirectly matching the D-cut surfaces against each other. The fixing member 18 has inner and outer bearing surfaces parallel to each other that bear the D-cut surfaces D1 and the D-cut surfaces D2, respectively.

For assembling the probe unit 3, the D-cut surfaces D1 of the probe 7 are inserted onto the corresponding bearing surfaces of the fixing member 18 and fixed in position. Then, the fixing member 18 is fitted into the handle portion 5 to have the corresponding bearing surfaces mate with the D-cut surfaces D2, and is fixed in position. When the probe unit 3 is thus assembled, the marker 4 and the cutting surface 15 of the treatment portion 8a are disposed in facing relation to each other. Instead of the D-cut surfaces, a pin may be mounted on the probe 7 at a position corresponding to a node of ultrasonic vibrations, and the handle portion 5 may have the positional relationship established between the marker 4 and the cutting surface 15 of the treatment portion 8 based on the position of the pin.

The handle portion 5 is of a shape in which the diameter progressively increases from a smaller outside diameter on the distal-end side toward a larger outside diameter for connection to the grip portion 2, so that the outer circumferential surface of the handle portion 5 smoothly blends into the outer circumferential surface of the grip portion 2. Normally, in a case where the ultrasonic treatment tool 1 is used in surgery or the like, it is used simultaneously and in combination with another medical device such as an endoscope. Therefore, a plurality of devices are present in a small area at a single treatment target site. The distal-end portion of the handle portion 5 is thus of a tapered shape to prevent the devices from physically interfering with each other when they are operated. In addition, the endoscope has an observational field of vision for observing the treatment portion 8 of the probe 7 and the treatment target site from the rear side, i.e., the proximal-end side of the probe. The tapered distal-end portion of the handle portion 5 is therefore effective to prevent part of the handle portion 5 from being included in the observational field of vision, making the necessary observational field of vision sufficiently large.

Next, the protrusion, i.e., first protrusion, 5a of the handle portion 5 will be described hereinafter.

As illustrated in FIG. 1, the handle portion 5 includes the protrusion 5a at an end portion thereof held against the grip portion 2. As illustrated in FIG. 2A, the protrusion 5a is formed as a flange-shaped ridge projecting radially outwardly into a larger diameter from part of the proximal-end side of the handle portion 5 fully along the circumference thereof. The protrusion 5a may not necessarily be of a larger diameter fully along the circumference of the handle portion 5, and may be present only in a location where it is engaged by a finger.

As illustrated in FIG. 1, the marker 4 extends from the protruding apex of the protrusion 5a toward the grip portion 2. The portion of the protrusion 5a that extends from the protruding apex of the protrusion 5a into contact with the grip portion 2 is constricted into a curved-surface shape. As the protrusion 5a is constricted into a curved-surface shape with a reduced diameter, the marker 4 is raised into a three-dimensional shape. The marker 4 has a distal end extending onto the grip portion 2. According to the present embodiment, the end of the curved-surface shape of the protrusion 5a and the distal end of the grip portion 2 have the same outside diameter so that they provide one step-free surface. Since there is no step between the protrusion 5a and the grip portion 2, the surgeon feels the marker 4 clearly raised, at the time of touching the marker 4.

The protrusion 5a not only functions to engage a finger or palm to make the grip portion 2 easy to grip, but also functions as a non-slip region or a support region for a finger or palm at the time of applying a force in a longitudinal direction, when the surgeon grips the grip portion 2 in first through fourth grip modes to be described hereinafter. The non-slip function of the protrusion 5a is useful as the surgeon may grip the grip portion 2 with a wet hand in surgery. Moreover, the curved surface of the rising portion of the protrusion 5a makes it easy for a finger to touch and engage the protrusion 5a.

The marker 4 that is of a raised or protrusive shape has been described. However, in a case where the protrusion 5a is included in the handle portion 5, a cut or a recess may be formed, instead of the protrusive marker 4, in the outer circumferential surface of the protrusion and may be used instead of the marker. The cut or the recess should preferably have its bottom colored for making itself visually recognizable.

When the marker 4 and the handle portion 5 are formed by the two-color injection molding described hereinbefore, only the marker 4 is integrally formed with the handle portion 5 according to the illustrated embodiment. According to another example, as illustrated in FIG. 2B, a ring-shaped member 5b including the marker 4 may be formed in a color different from the handle portion 5. The ring-shaped member 5b is of a shape similar to the end portion of the handle portion 5 that has the curved-surface shape described hereinbefore, and is fixedly fitted over the proximal-end side of the protrusion 5a of the handle portion 5. The end portion of the ring-shaped member 5b that has the curved-surface shape is formed such that it provides one surface together with the outer circumferential surface of the distal end of the grip portion 2 at the time the ring-shaped member 5b is connected to the grip portion 2.

Conversely, the ring-shaped member 5b may be angularly movably mounted on the distal-end side of the grip portion 2, i.e., the side thereof that fits in the handle portion 5. The ring-shaped member 5b and the handle portion 5 have recessed and projected portions that fit with each other. These recessed and projected portions are related to each other in advance such that when the handle portion 5 and the grip portion 2 are connected to each other, the marker 4 and the treatment surface of the treatment portion 8 face each other across the central axis of the probe 7.

For assembling the ultrasonic treatment tool 1, when the handle portion 5 and the grip portion 2 are connected to each other, the recessed and projected portions of the ring-shaped member 5b and the handle portion 5 are fitted with each other while the ring-shaped member 5b on the distal-end side of the grip portion 2 is being turned. With the recessed and projected portions fitted with each other, the marker 4 is related to the orientation of the treatment surface of the treatment portion 8. With this arrangement, the marker 4 may be disposed on the grip portion 2.

According to a connecting method, the probe 7 and the ultrasonic transducer 10 are connected by screwing under a tightening pressure using a torque wrench. With a simple screwed structure, the handle portion 5 may not necessarily be fixed to the grip portion 2 at a constant position due to manufacturing errors or the like. Therefore, if the marker 4 is fixed to the grip portion 2, then it is assumed that the marker 4 may be shifted from an initially established position. However, as the marker 4 is angularly movable, even if the marker 4 is shifted out of position upon being fixed, it can appropriately be corrected in position so as to match the orientation of the treatment surface of the treatment portion 8 when the handle portion 5 is connected to the grip portion 2.

The ring-shaped member 5b is available in a plurality of different colors including orange, blue, and yellow. Different colors associated with probe diameters and types of first through third treatment portions to be described hereinafter may be assigned to the ring-shaped member 5b. In other words, the ring-shaped member 5b including the marker 4 of the handle portion 5 is available in different colors associated with probe diameters and types of first through third treatment portions. In a case where the probe unit 3 according to the present embodiment is of the disposable type, it is individually sterilized and packaged prior to use. Therefore, the instant the probe unit 3 is taken out of the package, it is no longer reusable. In other words, once the package is opened by mistake, the probe unit 3 should be discarded even if it has been kept in storage as an unused item. When the surgeon or the like prepares a probe unit 3 for surgery, the surgeon is less likely to unseal the package in error by easily and appropriately identifying the probe unit 3 that has a treatment portion suitable for the application based on the color of the ring-shaped member 5b that is different from the other colors.

Next, the treatment portion 8 on the distal end of the probe 7 will be described hereinafter.

FIG. 2D is a view of the appearance, as viewed from one side, of a first treatment portion including a plurality of grooves. FIG. 2E is a view of the appearance, as viewed from one side, of a second treatment portion including a plurality of step-like projections. FIG. 2F is a diagram illustrating a reference side of a cutting surface of the second treatment portion. FIG. 2G is a view of the appearance, as viewed from one side, of a third treatment portion including a hook-shaped projection.

A first treatment portion denoted by 8a illustrated in FIG. 2D has a cutting surface 15 on a side surface of the probe 7. The cutting surface 15 is bent at a slight angle with respect to the longitudinal directions of the probe 7 for easy use.

The cutting surface 15 has a plurality of grooves defined therein that extend in directions transverse to the longitudinal directions of the probe 7, e.g., in directions perpendicular or oblique thereto. In a case where grooves are to be obliquely formed in the cutting surface 15, they may be formed in mutually transverse directions in a cross-hatched pattern. The grooves thus formed in the cutting surface 15 produce a number of edges. The cutting surface 15 of the first treatment portion 8a cuts a treatment target in moving directions m in which it reciprocates along the directions of ultrasonic vibrations, and is pressed to perform hammering based on vibrations, thereby cutting the treatment target in a cutting direction C laterally of the probe 7. The first treatment portion 8a has an excellent function to make the cut area into a flat surface. The cutting surface 15 is set in a position opposite the marker 4, i.e., facing the marker 4, across the central axis of the probe 7.

A second treatment portion denoted by 8b illustrated in FIG. 2E includes a cutting surface 16 formed on a front surface of the distal end of the probe 7. The second treatment portion 8b is of a rectangular shape or a polygonal shape as viewed from a front projected surface. The rectangular shape may be an oblong having a preset ratio of vertical and horizontal dimensions, for example. The second treatment portion 8b that is of the oblong with the preset ratio is able to form holes in various rectangular shapes and sizes by repeatedly cutting treatment targets while changing its orientation in vertical and horizontal directions. The projected surface, i.e., cutting surface, may be of any shape including an elliptical shape, a track shape, a star shape, other than the rectangular shape or the polygonal shape.

The cutting surface 16 includes a reference side 16a that represents one side, i.e., reference side, set as a given reference, in forming the rectangular or polygonal cutting surface. In FIG. 2F, if the cutting surface 16 is in a position that is opposite the marker 4 across the central axis of the probe 7, with the marker 4 being disposed on an upper side, then the reference side 16a of the cutting surface 16 is disposed on a lower side.

The cutting surface 16 of the second treatment portion 8b is of a shape protruding in a plurality of steps in thicknesswise directions. The cutting surface 16 cuts a treatment target in a moving direction m in which it drives in a direction of ultrasonic vibrations, i.e., cuts by way of hammering, thereby cutting the treatment target in a cutting direction C forward of the probe 7. The second treatment portion 8b is not limited to the stepwise projected shape, and may have a cutting surface transverse to the directions of ultrasonic vibrations for similarly cutting a treatment target though it has a different cutting efficiency. The second treatment portion 8b is excellent in its function to make a hole in a bone or the like.

If the cutting surface 16 is of a rectangular shape, then its direction is set with respect to the marker 4 by aligning the marker 4 with any one of the shorter or longer sides thereof as a reference side. If the cutting surface 16 is of a polygonal shape, then the marker 4 may be aligned with any one side thereof. If the cutting surface 16 is of an elliptical shape or an oval shape, i.e., a track shape, other than a true circular shape, then the orientation of the cutting surface 16 can be recognized by simply looking at the marker 4, providing the directions of vertical and horizontal dimensions, i.e., longer and shorter sides, thereof are set with respect to the marker 4.

A third treatment portion denoted by 8c illustrated in FIG. 2G has a cutting surface 17 on a side of the probe 7. The cutting surface 17 has a hook-shaped protrusion. When the cutting surface 17 is pressed against a treatment target, the vibrating protrusion cuts the treatment target in a cutting direction C laterally of the probe 7 by way of hammering and scraping. The third treatment portion 8c is excellent in its cutting function compared with the first treatment portion 8a, and is selected for a larger amount of biotissue to be cut off the treatment target. The marker 4 is positioned opposite the cutting surface 17 across the central axis of the probe 7 and is set in alignment with the orientation of the distal end of the hook of the protrusion.

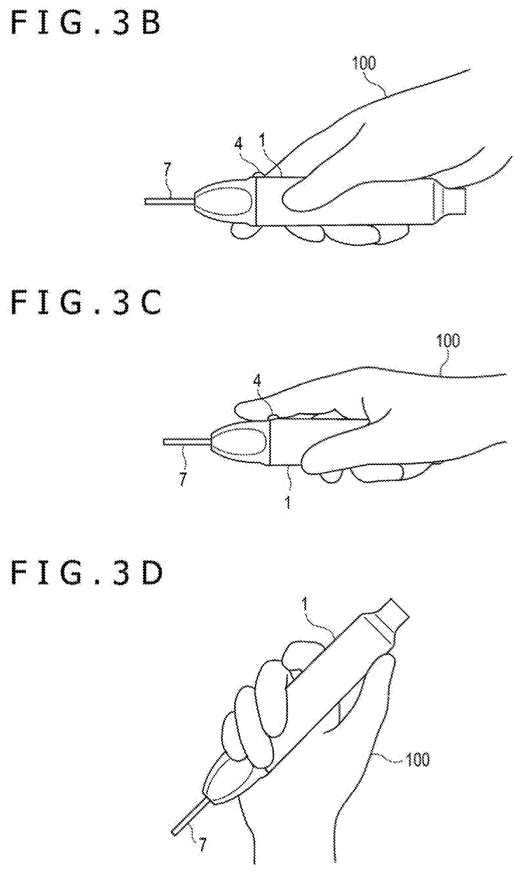

Next, gripped states of the ultrasonic treatment tool 1 will be described hereinafter with reference to FIG. 3A, FIG. 3B, FIG. 3C, and FIG. 3D. FIG. 3A illustrates a first gripped state in which the ultrasonic treatment tool 1 is gripped in the way a normal pen or the like is gripped. This gripped fashion is suitable for a treatment portion having a cutting surface on a side of the probe 7, i.e., the treatment portion 8a illustrated in FIG. 2D or the like. Particularly, the first gripped state allows the surgeon to finely adjust the way in which the cutting surface is pressed against a treatment target by pressing the ultrasonic treatment tool 1 with the wrist and fingers of a hand 100. The first gripped state makes it easy to move the ultrasonic treatment tool 1 in directions along the cutting surface, and is suitable for finely treating the treatment target in a finishing stage, for example. The surgeon can direct the cutting surface downwardly by gripping the ultrasonic treatment tool 1 such that the marker 4 is in contact with the index finger or is seen between the index finger and the thumb.

FIG. 3B illustrates a second gripped state that represents a gripped fashion often used when the grip of a racket or a tool is gripped. This gripped fashion is suitable for a treatment portion having a cutting surface on a side of the probe 7. This gripped fashion allows the surgeon to apply forces to the cutting surface in its entirety by pressing the palm of a hand 100 against the ultrasonic treatment tool 1. The second gripped state is suitable for pressing the entire cutting surface for cutting a larger amount of biotissue off the treatment target. The surgeon can direct the cutting surface downwardly by gripping the ultrasonic treatment tool 1 such that the marker 4 is seen between the index finger and the thumb.

FIG. 3C illustrates a third gripped state that represents a gripped fashion often used when a cutting tool, i.e., a carving knife, a slender kitchen knife, or the like, is gripped. This gripped fashion is suitable for a treatment portion having a cutting surface on a side of the probe 7. This gripped fashion allows the surgeon to apply forces to the cutting surface in its entirety by pressing the palm of a hand 100 against the grip portion 2, and makes it easy to move the treatment portion back and forth along the longitudinal directions. Specifically, the third gripped state is suitable for forming grooves or the like in a treatment target by repeatedly cutting the treatment target in the longitudinal directions. The third gripped state is also able to increase an amount of biotissue to be cut off the treatment target by pressing the cutting surface with the palm of the hand. Furthermore, the surgeon can press the distal-end side of the cutting surface locally by applying forces to the fingertip of the index finger. The surgeon can direct the cutting surface downwardly by holding the index finger in contact with the marker 4.

FIG. 3D illustrates a fourth gripped state that is referred to as an underhand grip, which allows the surgeon to push the ultrasonic treatment tool 1 downwardly with its own body weight as well as the arm muscles. This gripped fashion is suitable for a treatment portion having a cutting surface on a front surface of the distal end of the probe 7. The fourth gripped state allows the surgeon to apply forces in the longitudinal directions with the wrist or arm muscles. The fourth gripped state is suitable for increasing an amount of biotissue to be cut off the treatment target by pressing the cutting surface in its entirety and also for use with a hard treatment target. Providing the cutting surface is of a rectangular shape, the surgeon can direct the reference side downwardly by gripping the ultrasonic treatment tool 1 such that the marker 4 is felt by the little finger at its middle phalanx.

As described hereinbefore, the marker 4 according to the present embodiment is disposed on the handle portion 5 adjacent to the grip portion 2. Therefore, the surgeon can confirm the orientations in which the cutting surfaces 15 through 17 of the treatment portion 8 are directed simply by looking at its own hand. Since the handle portion 5 is integral with the marker 4 and the probe 7 as being positionally related thereto, the orientations of the cutting surfaces 15 through 17 and the marker 4 are aligned with each other regardless of the fixed position in which the handle portion 5 and the grip portion 2 are connected to each other.

Furthermore, inasmuch as the surgeon can grasp the direction in which the cutting surface of the ultrasonic treatment tool 1 is oriented by looking at the hand, when the surgeon regripped the ultrasonic treatment tool 1 from the third gripped state illustrated in FIG. 3C into the first gripped state illustrated in FIG. 3A, for example, the surgeon can keep the orientation of the cutting surface as before based on the marker 4. Therefore, the surgeon can immediately start a treatment. In an example of joint surgery, for example, in which one surgeon operates both an arthroscope and a treatment tool, if the treatment tool is free of the marker 4 according to the present embodiment and the surgeon cannot keep the orientation of the cutting surface based on the marker 4, then the surgeon is required to reorient the cutting surface 15 by seeing a screen captured by the arthroscope.

According to the present embodiment, however, as the marker 4 that indicates the orientation of the cutting surface 15 enables the surgeon to confirm the orientation of the cutting surface 15 at the hand, the surgeon can keep the orientation of the cutting surface as before when the surgeon has regripped the treatment tool. According to the present embodiment, therefore, the surgeon can start a cutting treatment simply by confirming the orientation of the cutting surface on the screen captured by the arthroscope. Thus, the working load on the surgeon is reduced, and the period of time required for the surgery is shortened. The shorter period of time required for the surgery is effective to reduce the physical burgeon on the patient and also to reduce the mental fatigue and physical fatigue on the surgeon.

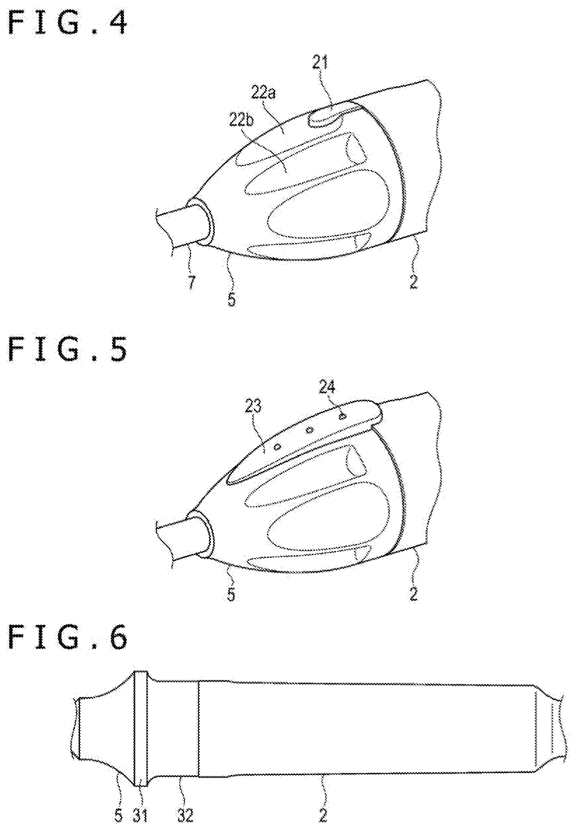

Next, a first modification of the marker will be described hereinafter with reference to FIG. 4. FIG. 4 is a view illustrating a marker 21 according to the first modification on the ultrasonic treatment tool.

A handle portion 5 is of a tapered shape whose outside diameter is progressively reduced from the rear end thereof toward the distal-end side where the proximal-end portion of the probe 7 is fixed. The marker 21 according to the first modification is of a shape that extends short and is raised slightly from the proximal end toward distal-end side of the handle portion 5. The marker 21 is formed in a color different from the handle portion 5 and the grip portion 2.

In this example, the handle portion 5 has on its outer circumferential surface an alternate array of flat surfaces 22a and grooves 22b. The marker 21 has a distal-end portion extending onto one of the flat surfaces 22a. Specifically, the distal-end portion of the marker 21 has a peripheral edge formed as if its surrounding region were cut off downwardly flatwise, making the marker 21 raised more on the flat surface 22a than on the proximal-end side thereof. Consequently, the surgeon can confirm the marker 21 more easily when contacting the marker 21 with a finger. The marker 21 protrudes to a low height from the surface of the grip portion 2, so that the marker 21 does not present an obstacle to fingers when the grip portion 2 is turned.

Next, a second modification of the marker will be described hereinafter with reference to FIG. 5. FIG. 5 is a view illustrating a marker 23 according to the second modification on the ultrasonic treatment tool.

The marker 23 according to the second modification is of a shape that extends longer than the marker 21 described hereinbefore and is raised slightly from the proximal end toward distal-end side of the handle portion 5. The marker 23 has a plurality of surface irregularities such as small-size protrusions 24 thereon. These protrusions 24 are arranged in an array and may be of a size large enough to be confirmed for their presence when contacted by a finger. The marker 23 is formed in a color different from the handle portion 5 and the grip portion 2. The marker 23 is easily visually recognizable, and functions as a finger rest doubling as a non-slip member if the grip portion 2 is gripped in the first gripped state described hereinbefore and illustrated in FIG. 3A in which the ultrasonic treatment tool 1 is gripped like a pen.

Next, a second protrusion 31 on the handle portion 5 of the ultrasonic treatment tool will be described hereinafter with reference to FIG. 6. In this example, the second protrusion 31 is disposed in a position shifted toward the distal-end side from the protrusion 5a on the handle portion 5 described hereinbefore. A flat portion 32 that is of the same diameter as the grip portion 2 is disposed between the second protrusion 31 and the grip portion 2 on the proximal-end side of the handle portion 5. The flat portion 32 is a portion to be gripped as with the grip portion 2, and is equivalent to an extended length of the grip portion 2.

An example in which the grip portion 2 is gripped as an underhand grip as illustrated in FIG. 3D will be described hereinafter. When the surgeon turns the grip portion 2 radially with the thumb and the index finger, depending on the size of the hand of the surgeon, if the grip portion 2 is longer, the surgeon may find it easier to operate the grip portion 2 without having the little finger or the like get caught. With the protrusion disposed in a changed position on the handle portion 5, the surgeon can select a grip portion size that matches the hand of the surgeon. The second protrusion 31 operates in the same manner and offers the same advantages as the protrusion 5a described hereinbefore.

As illustrated in FIG. 3A described hereinbefore, in a case where the grip portion 2 is gripped like a pen and the treatment portion 8 is pushed forwardly, the second protrusion 31 functions as a stopper for the fingers of the surgeon. Therefore, when the surgeon performs a treatment, the protrusion 31 is effective to prevent the hand from slipping off the grip portion 2. The marker 4 as an identifier that is disposed on the second protrusion 31 allows the surgeon to confirm the marker 4 with ease and grasp the orientation of the cutting surface because the marker 4 is near the position where the fingers are of necessity engaged by the second protrusion 31.

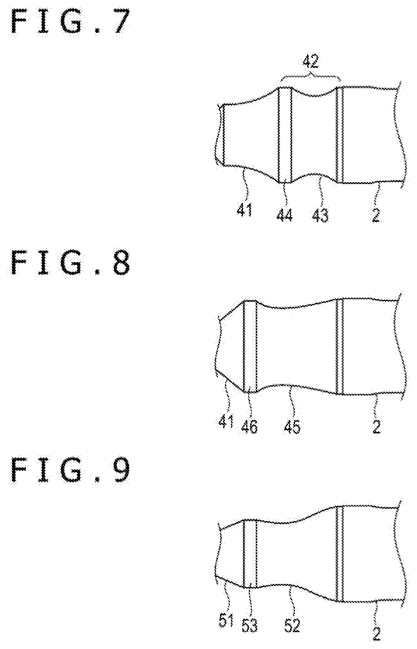

Next, a protrusion formed by a first constricted portion on a handle portion will be described hereinafter with reference to FIG. 7. FIG. 7 is a view illustrating a protrusion 44 formed by a first constricted portion 43 on a handle portion 41.

The protrusions 5a and 31 on the handle portion 5 described hereinbefore are formed by increasing the outside diameter of the grip portion 2 to make it easy for the fingers to engage the protrusions 5a and 31. However, in a case where a treatment is carried out in an environment near another medical device such as an endoscope, or in a case where a treatment is carried out at a position near a body of a patient, the protrusion formed by increasing the outside diameter of the grip portion 2 may possibly interfere with the other device or the patient's body, tending to impede the treatment. To avoid the shortcoming, at least one recessed constriction is formed on the handle portion 5 for achieving the same operation and advantages as with the protrusions described hereinbefore.

As illustrated in FIG. 7, the handle portion 41 has a region 42 that has a diameter equivalent to the outside diameter of the grip portion 2. A recess as the first constricted portion 43 is formed in the region 42. The recess thus formed produces the protrusion 44 that is of the same outside diameter as the grip portion 2, but has an essentially protrusive function.

The protrusion 44 that is formed by the first constricted portion 43 on the handle portion 41 as described hereinbefore operates in the same manner and offers the same advantages as the protrusions 5a and 31 described hereinbefore. Particularly, in a space where the surgical field is small because it is adjacent to another medical device or near the body of the patient, the protrusion 44 does not interfere with the other medical device, allowing the treatment to be carried out. In addition, since the protrusion is formed by constricting part of the handle portion 41, the position where the grip portion 2 is to be gripped can be recognized intuitively. Although not illustrated, the marker 4 described hereinbefore may be disposed on a side of the protrusion 44 that extends toward the first constricted portion 43 or on a side of the protrusion that extends toward the distal-end side of the handle portion 41.

Next, a protrusion formed by a second constricted portion on a handle portion will be described hereinafter with reference to FIG. 8. FIG. 8 is a view illustrating a protrusion 46 formed by a second constricted portion 45 on a handle portion 41.

The second constricted portion 45 is formed to the same depth as, but to a length larger than, the first constricted portion 43 described hereinbefore. Therefore, the protrusion 46 on the handle portion 41 is disposed in a position shifted toward the distal-end side from the protrusion 44. Since the protrusion 46 on the handle portion 41 is disposed in the position shifted toward the distal-end side, the ultrasonic treatment tool has a large essential gripping area including the grip portion 2. Therefore, it is possible for the surgeon to select a grip portion size matching the way in which the surgeon grips the ultrasonic treatment tool or the size of the hand of the surgeon. The protrusion 46 formed by the second constricted portion 45 operates in the same manner and offers the same advantages as the protrusion 44 formed by the first constricted portion 43.

Next, a protrusion formed by a third constricted portion on a handle portion will be described hereinafter with reference to FIG. 9. FIG. 9 is a view illustrating a protrusion 53 formed by a third constricted portion 52 on a handle portion 51.

The handle portion 51 is of a conical shape that is tapered toward the probe 7, as illustrated in FIG. 4. The handle portion 51 includes the third constricted portion 52 that is of the same length as the second constricted portion 45 described hereinbefore. Therefore, the protrusion 53 on the handle portion 51 is an annular protrusion that is of a diameter smaller than the outside diameters of the proximal-end portion of the handle portion 51 and the grip portion 2.

On the handle portion 51, the third constricted portion 52 is smaller in diameter on the distal-end side than on the rear end, and the protrusion 53 is also smaller in diameter. The shapes of the third constricted portion 52 and the protrusion 53 lend themselves to the way in which the ultrasonic treatment tool is gripped like a pen, as illustrated in FIG. 3A. Specifically, when the fingers are placed on the handle portion 51 in the way a pen is gripped, the area surrounded by the fingers, i.e., the thumb, the index finger, and the middle finger, is smaller than the outside diameter of the grip portion 2, and the grip portion 2 is in contact with the bases of the thumb and the index finger. Consequently, as the area surrounded by the fingers, i.e., the thumb, the index finger, and the middle finger, is smaller, the surgeon can grip the ultrasonic treatment tool 1 stably, and can perform fine fingering as if writing letters. For example, the surgeon can finish holes or grooves formed in orthopedic surgery or the like with fine fingering.

The disclosed technology is not limited to the embodiments described hereinbefore, and various modifications may be made therein without departing from the scope of the invention when it is reduced to practice. The embodiments may be appropriately combined as much as possible, and the combinations offer combined advantages. Furthermore, the embodiments include inventions in various stages, and various inventions can be extracted by appropriately combining a plurality of components that are disclosed.

In sum, one aspect of the disclosed technology is directed to a probe unit of an ultrasonic treatment tool that comprises an ultrasonic probe having respective opposed distal and proximal end sides in a longitudinal direction and configured to transmit ultrasonic vibrations. A treatment portion is attached to the distal end side of the ultrasonic probe in the longitudinal direction. The treatment portion includes a cutting surface of a shape having directionality. A handle portion having a cutting portion identifier that is engaged with an outer circumference of the proximal end side of the ultrasonic probe. The cutting portion identifier is configured to indicate a position of a reference side representing a direction in which the cutting surface is oriented and/or included in the cutting surface.

In the probe unit of the ultrasonic treatment tool, at least one flat surface is formed on an outer circumferential surface of the proximal-end portion of the ultrasonic probe and a fixed position and a fixed direction on the handle portion are defined based on the at least one flat surface. The cutting portion identifier on the handle portion and the reference side representing the direction in which the cutting surface is oriented and/or included in the cutting surface are disposed on respective sides facing one another across a central axis of the ultrasonic probe. The cutting portion identifier includes a protrusion or a groove formed on the handle portion in a color different from the handle portion. The handle portion includes a protrusion defined by a flange on an outer circumferential surface of the proximal-end side of the handle portion. The cutting portion identifier extends rearwardly into a three-dimensional shape from an apex of the protrusion. The cutting portion identifier is separate from the handle portion and a ring-shaped member including the cutting portion identifier and the protrusion is mounted on an outer surface of a rear end of the handle portion.

The ultrasonic treatment tool includes an ultrasonic transducer for producing ultrasonic vibrations and a grip portion being detachably mounted on a rear-end side of the handle portion. The protrusion of the handle portion is larger in outside diameter than the grip portion and is of a curved shape that is progressively reduced in diameter from the apex of the protrusion toward the rear end of the handle portion and that has the same outside diameter as the grip portion, providing a step-free surface blending into an outer surface of a distal end of the grip portion. In the probe unit of the ultrasonic treatment tool, the handle portion defines a shape having a progressively smaller outside diameter from a rear-end side thereof toward the distal-end side to which a proximal-end portion of the ultrasonic probe is attached. The handle portion includes at least one constricted portion having a length such that the shape having the progressively smaller outside diameter. The handle portion includes at least one constricted portion having a length in a location from a rear-end side thereof toward a distal-end side to which a proximal-end portion of the ultrasonic probe is attached. The constricted portion defines a shape having a progressively smaller outside diameter from a distal-end side thereof.

Another aspect of the disclosed technology is directed to an ultrasonic treatment tool comprises a probe unit configured to be used with the ultrasonic treatment tool for treating bones and soft bones using ultrasonic vibrations. A grip portion is detachably mounted on a proximal-end side of the probe unit. The grip portion contains therein an ultrasonic transducer for producing ultrasonic waves and transmitting ultrasonic vibrations to the probe unit. The probe unit includes an ultrasonic probe having respective opposed distal and proximal end sides in a longitudinal direction and configured to transmit the ultrasonic vibrations. A treatment portion is attached to the distal end side of the ultrasonic probe in longitudinal direction. The treatment portion includes a cutting surface of a shape having directionality. A handle portion having a cutting portion identifier that is engaged with an outer circumference of the proximal end side of the ultrasonic probe. The cutting portion identifier is configured to indicate a position of a reference side representing a direction in which the cutting surface is oriented and/or included in the cutting surface.

In the ultrasonic treatment tool, a distal end of the grip portion and a proximal end of the probe unit are attached to one another. The grip portion defines a hollow cylindrical shape and has a diameter equal to or smaller than a diameter of a proximal end of the probe unit. In the ultrasonic treatment tool, the cutting portion identifier is positioned on a proximal-end side of the handle portion and extends toward the grip portion.

While various embodiments of the disclosed technology have been described above, it should be understood that they have been presented by way of example only, and not of limitation. Likewise, the various diagrams may depict an example schematic or other configuration for the disclosed technology, which is done to aid in understanding the features and functionality that can be included in the disclosed technology. The disclosed technology is not restricted to the illustrated example schematic or configurations, but the desired features can be implemented using a variety of alternative illustrations and configurations. Indeed, it will be apparent to one of skill in the art how alternative functional, logical or physical locations and configurations can be implemented to implement the desired features of the technology disclosed herein.

Although the disclosed technology is described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the disclosed technology, whether or not such embodiments are described and whether or not such features are presented as being a part of a described embodiment. Thus, the breadth and scope of the technology disclosed herein should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent.

Additionally, the various embodiments set forth herein are described in terms of exemplary schematics, block diagrams, and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular configuration.

What is claimed is:

1. A probe unit of an ultrasonic treatment tool, comprising:
an ultrasonic probe having respective opposed distal and proximal end sides in a longitudinal direction and configured to transmit ultrasonic vibrations;
a treatment portion being attached to the distal end side of the ultrasonic probe in the longitudinal direction, the treatment portion includes a cutting surface of a shape having directionality; and
a handle portion having a cutting portion identifier that being engaged with an outer circumference of the proximal end side of the ultrasonic probe wherein
the cutting portion identifier configured to indicate a position of a reference side representing a direction in which the cutting surface is oriented and/or included in the cutting surface.

2. The probe unit of the ultrasonic treatment tool of claim 1, wherein
at least one flat surface is formed on an outer circumferential surface of the proximal-end portion of the ultrasonic probe and wherein a fixed position and a fixed direction on the handle portion are defined based on the at least one flat surface; and
the cutting portion identifier on the handle portion and the reference side representing the direction in which the cutting surface is oriented and/or included in the cutting surface are disposed on respective sides facing one another across a central axis of the ultrasonic probe.

3. The probe unit of the ultrasonic treatment tool of claim 1, wherein
the cutting portion identifier includes a protrusion or a groove formed on the handle portion in a color different from the handle portion.

4. The probe unit of the ultrasonic treatment tool of claim 1, wherein
the handle portion includes a protrusion defined by a flange on an outer circumferential surface of the proximal-end side of the handle portion; and
the cutting portion identifier extends rearwardly into a three-dimensional shape from an apex of the protrusion.

5. The probe unit of the ultrasonic treatment tool of claim 4, wherein
the cutting portion identifier is separate from the handle portion and a ring-shaped member including the cutting portion identifier and the protrusion is mounted on an outer surface of a rear end of the handle portion.

6. The probe unit of the ultrasonic treatment tool of claim 4, wherein
the ultrasonic treatment tool includes an ultrasonic transducer for producing ultrasonic vibrations and a grip portion being detachably mounted on a rear-end side of the handle portion; and
the protrusion of the handle portion is larger in outside diameter than the grip portion and is of a curved shape that is progressively reduced in diameter from the apex of the protrusion toward the rear end of the handle portion and that has the same outside diameter as the grip portion, providing a step-free surface blending into an outer surface of a distal end of the grip portion.

7. The probe unit of the ultrasonic treatment tool of claim 1, wherein
the handle portion defines a shape having a progressively smaller outside diameter from a rear-end side thereof toward the distal-end side to which a proximal-end portion of the ultrasonic probe is attached.

8. The probe unit of the ultrasonic treatment tool of claim 7, wherein
the handle portion includes at least one constricted portion having a length such that the shape having the progressively smaller outside diameter.

9. The probe unit of the ultrasonic treatment tool of claim 1, wherein
the handle portion includes at least one constricted portion having a length in a location from a rear-end side thereof toward a distal-end side to which a proximal-end portion of the ultrasonic probe is attached; and
the constricted portion defines a shape having a progressively smaller outside diameter from a distal-end side thereof.

10. An ultrasonic treatment tool comprising:
a probe unit configured to be used with the ultrasonic treatment tool for treating bones and soft bones using ultrasonic vibrations; and
a grip portion being detachably mounted on a proximal-end side of the probe unit, the grip portion contains therein an ultrasonic transducer for producing ultrasonic waves and transmitting ultrasonic vibrations to the probe unit, wherein
the probe unit includes
an ultrasonic probe having respective opposed distal and proximal end sides in a longitudinal direction and configured to transmit the ultrasonic vibrations,
a treatment portion being attached to the distal end side of the ultrasonic probe in longitudinal direction, the treatment portion includes a cutting surface of a shape having directionality,
a handle portion having a cutting portion identifier that being engaged with an outer circumference of the proximal end side of the ultrasonic probe wherein
the cutting portion identifier configured to indicate a position of a reference side representing a direction in which the cutting surface is oriented and/or included in the cutting surface.

11. The ultrasonic treatment tool of claim 10, wherein
a distal end of the grip portion and a proximal end of the probe unit are attached to one another.

12. The ultrasonic treatment tool of claim 10, wherein
the grip portion defines a hollow cylindrical shape and has a diameter equal to or smaller than a diameter of a proximal end of the probe unit.

13. The ultrasonic treatment tool of claim 10, wherein
the cutting portion identifier is positioned on a proximal-end side of the handle portion and extends toward the grip portion.
